(19) United States
(12) Patent Application Publication
(10) Pub. No.: US 2021/0386640 A1
(43) Pub. Date: Dec. 16, 2021

(54) DISINFECTING COMPOSITION FOR TOPICAL USE

(71) Applicant: SOCIETE D'EXPLOITATION DE PRODUITS POUR LES INDUSTRIES CHIMIQUES SEPPIC, Paris Cedex 07 (FR)

(72) Inventors: Marie-Francoise CHIRAC, Paris (FR); Catherine BULCOURT, Paris (FR)

(21) Appl. No.: 17/284,380

(22) PCT Filed: Oct. 9, 2019
(86) PCT No.: PCT/FR2019/052392

§ 371 Date: Apr. 9, 2021

(30) Foreign Application Priority Data
Oct. 12, 2018 (FR) 1859467

(51) Int. Cl. A61K 8/44; A61K 8/73; A61Q 17/00

(52) U.S. Cl. CPC A61K 8/44; A61Q 17/005; A61K 8/73

(57) ABSTRACT

Disclosed is a disinfecting composition for topical use, including a surfactant mixture based on cocoyl glutamate and alkyl polyglycosides, at least one thickening and/or gelling agent, and at least one disinfectant.

### CROSS-REFERENCE TO RELATED APPLICATIONS

This application is the U.S. national phase of International Application No. PCT/FR2019/052392 filed Oct. 9, 2019 which designated the U.S. and claims priority to FR 1859467 filed Oct. 12, 2018, the entire contents of each of which are hereby incorporated by reference.

### BACKGROUND OF THE INVENTION

### Field of the Invention

The present invention relates to compositions for topical use, preferably used for hygienic disinfection and for disinfectant washing of the hands, mucous membranes and damaged parts of the skin.

The present invention finds application in the human and veterinary cosmetic and dermocosmetic, dermopharmaceutical and pharmaceutical fields.

### Description of the Related Art

The objective of hand disinfection, hand decontamination and more generally skin antisepsis is to prevent the transmission of microorganisms and viruses or to suppress their unwanted introduction into threatened and/or injured regions of the body or more sensitive regions. Compositions for hygienic disinfection and for disinfectant washing of the hands and sensitive parts of the skin must meet efficacy requirements, some of which are defined by standards. Various methods are possible for treating the hands after contamination.

Compositions for topical use for disinfectant washing and hygienic disinfection of the hands must be efficacious after acting for short periods (e.g. 30 seconds or 1 minute). For toxicological reasons, in addition to adequate efficacy, compatibility with human skin is in particular also necessary.

Commercially available handwashing compositions with disinfectant are usually liquid soaps containing alcohol, and/or surfactants, and ready-to-use wash lotions with other added biocidal agents.

Known compositions include, as biocidal agents, for example short-chain alcohols and excipients, regreasers, moisturizers and fragrances for improving skin compatibility and acceptance.

In order to increase the efficacy and obtain a residual effect intended to avoid a further increase in the number of microbes on the hands, the known compositions often also comprise additional agents such as biguanides (for example chlorhexidine), quaternary ammonium compounds (for example benzalkonium chloride), phenolic derivatives (for example ortho-phenylphenol) or carboxylic acids.

To increase the efficacy and obtain a residual effect intended to prevent a further increase in the number of microbes on the part of the skin treated, it is possible to seek to have available a foam comprising at least one disinfectant, the stability of which is high, viscous, so as to allow a greater contact time between the foam comprising the disinfectant with the surface to be treated.

Furthermore, formulations for foaming and disinfecting formulations for topical use require rapid foam formation when applied to the part of the body to be disinfected. Indeed, in the mind of the consumer, the formation of foam, in a rapid time, constitutes one of the pieces of evidence of the effectiveness of the application and the disinfection. The rate of formation of the foam (or expansion time), the volume of this foam, its stability and the pleasant sensations it affords are important parameters to be taken into account in the hope of commercial success of these formulations.

Foaming and disinfecting formulations for topical use comprise foaming surfactants, whether cationic, anionic, amphoteric or nonionic in nature. These surfactants are said to be amphiphilic since they consist of a hydrophilic part (or polar head), which is water-soluble, and of a lipophilic part (hydrophobic tail) which has affinity with oils and fats. This amphiphilic structure makes it possible both to dissolve fatty soiling and to remove it by a washing operation.

Anionic surfactants are historically the surfactants that have been used for preparing foaming formulations for topical use. Sulfate-based anionic surfactants constitute a class of surfactants that is frequently used on account of their good foaming properties. These surfactants to produce an aerated foam, the feel of which is not judged to be unpleasant by the consumer. However, these surfactants have the drawback of being sensitive to the degree of water hardness and to the presence of fatty soiling, which consequently leads to a reduction in the volume of foam initially generated by these formulations, but most of all to a reduction in the stability over time of this volume of foam.

To improve the stability of foams, those skilled in the art of detergency knows that they can combine foaming surfactants with one or more additives having the effect of increasing the stiffness of the gas cells forming the foam.

However, the foam-stabilizing additives are often poorly biodegradable and are occasionally toxic, which makes them noncompliant with the new environmental requirements and regulatory provisions, and for application to the skin.

It has been observed by the applicant that compositions based on a sodium salt of cocoyl glutamate and on mixtures of C₁₂-C₁₆ alkyl polyglucosides, and/or of alkyl polyglucosides the alkyl chain of which carries 4 to 7 carbon atoms, do not allow stable foam to be produced after addition of disinfectant.

There is therefore a real need to have a foaming composition which makes it possible to overcome the drawbacks of the foaming compositions of the prior art, that is to say which makes it possible in particular to prepare disinfecting foams for topical use, and to use such compositions for hygienic disinfection and for the disinfectant washing of the hands, mucous membranes, and damaged parts of the skin, in an efficacy approach.

### SUMMARY OF THE INVENTION

A solution of the present invention is a disinfecting composition (C_{D}) for topical use comprising, per 100% of its mass

a)—from 35% to 99.3% by mass of water, preferably from 37% to 98.7% by mass and even more preferentially from 40% to 97.6% by mass of water;
b)—from 0.5% to 40% by mass, preferably from 1% to 40% by mass, and even more preferentially from 2% to 40% by mass of at least one disinfectant;
c)—from 0.1% to 10% by mass, preferably from 0.1% to 8% by mass and even more preferentially from 0.2% to 8% by mass of at least one gelling agent and/or thickener (AG);
d)—from 0.1% to 15% by mass, preferably from 0.2% to 15% by mass, and even more preferentially from 0.2% to 12% by mass of a mixture (M₁) comprising, per 100% of its own mass:
(i)—from 50% to 99% by mass, preferably from 55% to 99% by mass, and even more preferentially from 57% to 99% by mass of a composition (C₁) comprising, per 100% of its mass:
(α)—from 65% to 90% by mass, preferably from 65% to 85% by mass and more preferentially from 65% to 80% by mass of at least one compound of formula (I):

R₁—C(═O)—NH—CH(COOH)—(CH₂)₂—COOH (I)

in acid, partially salified or totally salified form, wherein the group R₁—C(═O)— represents a linear or branched, saturated or unsaturated acyl radical including from 8 to 18 carbon atoms, and
(β)—from 10% to 35% by mass, preferably from 15% to 35% by mass and more preferentially from 20% to 35% by mass of at least one compound of formula (II):

R₁—C(═O)—OH (II)

in acid, partially salified or totally salified form, wherein the group R₁ is as defined for formula (I),
(ii)—from 1% to 50% by mass, preferably from 1% to 45% by mass and even more preferentially from 1% to 43% by mass of a composition (C₂) comprising, per 100% of its mass:
(γ)—from 14% to 80% by mass of a composition (C₃) or of a mixture of compositions (C₃), said composition (C₃) being represented by formula (III):

R₃—O-(G₃)ₚ-H (III)

wherein R₃ represents a linear or branched, saturated or unsaturated aliphatic radical including from 12 to 16 carbon atoms, G₃ represents the residue of a reducing sugar and p represents a decimal number greater than or equal to 1.05 and less than or equal to 5;
(δ)—from 20% to 80% of a composition (C₄) or of a mixture of compositions (C₄), said composition (C₄) being represented by formula (V):

R₄—O-(G₄)_{q}-H (V)

wherein R₄ represents a linear aliphatic radical, chosen from n-butyl (n-C₄H₉—), n-pentyl (n-C₅H₁₁—), n-hexyl (n-C₆H₁₃—) and n-heptyl (n-C₇H₁₅—) radicals, G₄ represents a reducing sugar residue and q represents a decimal number greater than or equal to 1.05 and less than or equal to 5.

It should be noted that the composition according to the invention will preferably be in foaming form.

In the context of the present invention, the term “compounds of formula (I) or (II) in acid, partially salified or totally salified form” means that one, several or all of the carboxyl functions present in one or other of said compounds of formula (I) or (II) is either in acid form (—COOH) or in salified form (—COO⁻M⁺). In the latter case, M⁺ represents a monovalent cation chosen from:
the ammonium cation,
monovalent cations of alkali metals, for example the sodium (Na⁺), potassium (K⁺) or lithium (Li⁺) cation,
   (hydroxyalkyl)ammonium, bis(hydroxyalkyl)ammonium or tris(hydroxyalkyl)ammonium cations wherein the hydroxyalkyl radical(s) include from 1 to 4 carbon atoms, for example 2-hydroxyethanammonium, 2-hydroxypropanammonium, bis(2-hydroxyethyl)ammonium and tris(2-hydroxyethyl)ammonium cations,
   (alkyloxyalkyl)ammonium, bis(alkyloxyalkyl)ammonium or tris(alkyloxyalkyl)ammonium cations wherein the alkyloxyalkyl radical(s) include from 2 to 6 carbon atoms, for example the 2-ethoxyethanammonium cation,
(hydroxyalkylaminoalkyl)ammonium, bis(hydroxyalkylaminoalkyl)ammonium or tris(hydroxyalkylaminoalkyl)ammonium cations wherein the hydroxyalkylaminoalkyl radical(s) include from 2 to 6 carbon atoms, for example the 2-hydroxyethylaminomethanammonium cation and the 2-hydroxyethylaminoethanammonium cation.

Depending on the case, the disinfecting composition (C_{D}) according to the invention may have one or more of the following characteristics:
the gelling agent and/or thickener (AG) is chosen from polysaccharides consisting of monosaccharide derivatives, polysaccharides consisting solely of monosaccharides, cellulose and cellulose derivatives, starches and linear or branched or crosslinked polyelectrolytes;
the composition (C₃) consists of a mixture of compounds represented by formulae (III₁), (III₂), (III₃), (III₄) and (III₅):

R₃—O-(G₃)₁-H (III₁),

R₃—O-(G₃)₂-H (III₂),

R₃—O-(G₃)₃-H (III₃),

R₃—O-(G₃)₄-H (III₄),

R₃—O-(G₃)ₛ-H (III₅),

in the respective molar proportions a₁, a₂, a₃, a₄ and a₅ such that:
the sum a₁+a₂+a₃+a₄+a₅ is equal to 1, and
the sum a₁+2a₂+3a₃+4a₄+5 as is equal to p;
the composition (C₄) consists of a mixture of compounds represented by formulae (V₁), (V₂), (V₃), (V₄) and (V₅):

R₄—O-(G₄)₁-H (V₁),

R₄—O-(G₄)₂-H (V₂),

R₄—O-(G₄)₃-H (V₃),

R₄—O-(G₄)₄-H (V₄),

R₄—O-(G₄)ₛ-H (V₅),

in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q;
the composition (C₂) comprises from 0% to 3% by mass of at least one alcohol of formula (IV):

R₃—OH (IV)

wherein R₃ is as defined for formula (III), and/or from 0% to 3% by mass of at least one alcohol of formula (VI):

R₄—OH (VI)

wherein R₄ is as defined for formula (V);
in formula (III) of the mixture (M₁) as defined above, G₃ and G₄, which may be identical or different, represent, independently of one another, a reducing sugar residue, glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose;
the compounds of formula (I) and of formula (II) are partially or totally salified in sodium salt or potassium salt form;
the mixture (M₁) comprises, per 100% of its mass, from 55% to 99% by mass of said composition (C₁) and from 1% by mass to 45% by mass of said composition (C₂), and more particularly from 57% to 99% by mass of said composition (C₁) and from 1% by mass to 43% by mass of said composition (C₂);
the composition (C₁) comprises, per 100% of its mass, from 65% to 90% by mass of one or more compounds of formula (I) and from 10% to 35% by mass of one or more compounds of formula (II); and more particularly from 65% to 85% by mass of one or more compounds of formula (I) and from 15% to 35% by mass of one or more compounds of formula (II);
in formulae (I) and (II), the group R₁—C(═O)— represents an acyl radical chosen from octanoyl, decanoyl, dodecanoyl, tetradecanoyl, hexadecanoyl, octadecanoyl, 9-octadecenoyl, 9,12-octadecadienoyl and 9,12,15-octadecatrienoyl radicals;
the composition (C₂) comprises per 100% of its mass:
   (γ)—a mass proportion of said composition (C₃) of greater than or equal to 14% and less than 70%, and
   (δ)—a mass proportion of said alcohol of formula (IV) of greater than or equal to 0% and less than or equal to 3%,
   (ε)—a mass proportion of said composition (C₄) of greater than or equal to 30% and less than or equal to 80%, and
   (η)—a mass proportion of said alcohol of formula (VI) of greater than or equal to 0% and less than or equal to 3%;
the composition (C₂) comprises, per 100% of its mass, a proportion by mass of said composition (C₃) greater than or equal to 14% and less than or equal to 65%, a proportion by mass of said alcohol of formula (IV) greater than or equal to 0% and less than or equal to 3%, a proportion by mass of said composition (C₄) greater than or equal to 35% and less than or equal to 80% and a proportion by mass of said alcohol of formula (VI) greater than or equal to 0% and less than or equal to 3%.
in formula (III), G₃ represents a reducing sugar residue chosen from glucose, xylose and arabinose residues;
in formula (III), p represents a decimal number greater than or equal to 1.05 and less than or equal to 2.5, preferably greater than or equal to 1.05 and less than or equal to 2.0, and even more preferentially greater than or equal to 1.25 and less than or equal to 2.0;
in formulae (III) and (IV) R₃ represents a linear alkyl radical chosen from n-dodecyl (n-C₁₂H₂₅—), n-tetradecyl (n-C₁₄H₂₉—) and n-hexadecyl (n-C₁₆H₃₂—) radicals;
in formula (V), G₄ represents a reducing sugar residue chosen from glucose, xylose and arabinose residues;
in formula (V), q represents a decimal number greater than or equal to 1.05 and less than or equal to 2.5, preferably greater than or equal to 1.05 and less than or equal to 2.0, and even more preferentially greater than or equal to 1.25 and less than or equal to 2.0;
in formulae (V) and (VI), R₄ represents a linear alkyl radical chosen from the n-hexyl (n-C₆H₁₃) and n-heptyl (n-C₇H₁₅) radicals; preferably, in formulae (V) and (VI), R₄ represents the heptyl (n-C₇H₁₅—) radical; according to another preferential embodiment, in formulae (V) and (VI), R₄ represents the n-hexyl (n-C₆H₁₃—) radical;
the composition (C₂) comprises a mixture of compositions (C₃) and compositions (C₄), said mixture comprising, per 100% of its mass:
   (γ₁)—from 13.6% to 44.4% by mass of a composition (C₃) represented by formula (III) wherein R₃ represents the (n-C₁₂H₂₅) radical,
   (γ₂)—from 5% to 16.25% by mass of a composition (C₃) represented by formula (III) wherein R₃ represents the n-tetradecyl (n-C₁₄H₂₉) radical, and
   (γ₃)—from 1.4% to 4.55% by mass of a composition (C₃) represented by formula (III) wherein R₃ represents the n-hexadecyl (n-C₁₆H₃₂) radical,
   (ε₁)—from 35% to 80% by mass of a composition (C₄) represented by formula (V) wherein R₄ represents the n-heptyl (n-C₇H₁₅) radical;
said composition (C₂) comprises a mixture of compositions (C₃) and compositions (C₄), said mixture comprising, per 100% of its mass:
   (γ₁)—from 13.6% to 44.4% by mass, more particularly from 17% to 44.4% by mass, of a composition (C₃) represented by formula (III) wherein R₃ represents the n-dodecyl (n-C₁₂H₂₅) radical,
   (γ₂)—from 5% to 16.25% by mass, more particularly from 6.25% to 16.25% by mass, of a composition (C₃) represented by formula (III) wherein R₃ represents the n-tetradecyl (n-C₁₄H₂₉) radical, and
   (γ₃)—from 1.4% to 4.55% by mass, more particularly from 1.75% to 4.55% by mass, of a composition (C₃) represented by formula (III) wherein R₃ represents the n-hexadecyl (n-C₁₆H₃₂) radical, and
   (ε₁)—from 35% to 80% by mass, more particularly from 35% to 75% by mass, of a composition (C₄) represented by formula (V) wherein R₄ represents the n-hexyl (n-C₆H₁₃) radical;
in formula (III), G₃ represents a reducing sugar residue chosen from glucose, xylose and arabinose residues, p represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₃ represents a linear alkyl radical chosen from n-dodecyl (n-C₁₂H₂₅), n-tetradecyl (n-C₁₄H₂₉) and n-hexadecyl (n-C₁₆H₃₂) radicals;
in formula (III), G₃ represents a glucose residue, p represents a decimal number greater than or equal to 1.05 and less than or equal to 2.5, and R₃ represents a linear alkyl radical chosen from n-dodecyl (n-C₁₂H₂₅), n-tetradecyl (n-C₁₄H₂₉) and n-hexadecyl (n-C₁₆H₃₂) radicals;
in formula (III), G₃ represents a xylose residue, p represents a decimal number greater than or equal to 1.05 and less than or equal to 2.5, and R₃ represents a linear alkyl radical chosen from n-dodecyl (n-C₁₂H₂₅) radicals, the n-tetradecyl (n-C₁₄H₂₉) radical and the n-hexadecyl (n-C₁₆H₃₂) radical;
in formula (V), G₄ represents a reducing sugar residue chosen from glucose, xylose and arabinose residues, q represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₄ represents an aliphatic alkyl radical chosen from n-hexyl (n-C₆H₁₃) radicals and the, n-heptyl (n-C₇H₁₅) radical;
in formula (V), G₄ represents a glucose residue, q represents a decimal number greater than or equal to 1.05 and less than or equal to 2.5, and R₄ represents an aliphatic alkyl radical chosen from n-hexyl (n-C₆H₁₃) and n-heptyl (n-C₇H₁₅) radicals;
in formula (V), G₄ represents a glucose residue, q represents a decimal number greater than or equal to 1.05 and less than or equal to 2.5, and R₄ represents the n-heptyl (n-C₇H₁₅) radical;
in formula (V), G₄ represents a xylose residue, q represents a decimal number greater than or equal to 1.05 and less than or equal to 2.5, and R₄ represents an aliphatic alkyl radical chosen from n-hexyl (n-C₆H₁₃) and n-heptyl (n-C₇H₁₅) radicals;
in formula (V), G₄ represents a xylose residue, q represents a decimal number greater than or equal to 1.05 and less than or equal to 2.5, and R₄ represents the n-heptyl (n-C₇H₁₅) radical;
the mass ratio:
   Δ=Mass of compound(s) of formula (I)/[Mass of composition (C₃)+Mass of composition (C₄)], is greater than or equal to 20/80 and less than or equal to 65/35, preferably greater than or equal to 25/75 and less than or equal to 65/35;
the mass ratio:
   Δ₁=Mass of the composition (C₃)/Mass of the composition (C₄) is greater than or equal to 20/80 and less than or equal to 70/30, preferably greater than or equal to 20/80 and less than or equal to 65/35, and even more preferentially greater than or equal to 25/75 and less than or equal to 65/35;
at least one gelling agent and/or thickener (AG) chosen from xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to ⅓ and less than or equal to 3/1;
at least one disinfectant is chosen from the elements of the group consisting of sorbic acid, sodium sorbate, potassium sorbate, dehydroacetic acid, sodium dehydroacetate, benzoic acid, sodium benzoate, potassium benzoate; 1-(2-ethylhexyl) glycerol; hydrogen peroxide; activated peroxides such as mixtures of hydrogen peroxide and sodium bicarbonate, mixtures of hydrogen peroxide and urea, mixtures of hydrogen peroxide and peracetic acid and mixtures of hydrogen peroxide and iron (Fenton's reagent); hydroperoxycarbonates; peracetic acid; sodium hypochlorite;
the disinfecting composition (C_{D}) comprises per 100% of its mass:
   a)—from 35% to 99.3% water;
   b)—from 0.5% to 40% by mass of at least one disinfectant chosen from the group consisting of hydrogen peroxide, sodium hypochlorite, potassium sorbate, sodium benzoate, 1-(2-ethylhexyl) glycerol,
   c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to ⅓ and less than or equal to 3/1;
   d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical.

The term “reducing sugar residue” denotes in the definition residues (G₃) and (G₄) of formulae (III) and (V) as defined previously, residues of saccharide derivatives without a glycoside bond established between an anomeric carbon and the oxygen of an acetal group, as defined in the reference publication: “Biochemistry, Daniel Voet/Judith G. Voet, p. 250, John Wyley & Sons, 1990.”

The oligomeric structures (G₃)ₚ and (G₄)_{q} may be in any isomeric form, whether it is optical isomerism, geometrical isomerism or regioisomerism; it may also represent a mixture of isomers.

In formula (III) as defined above, the group R₃ is linked to G₃ via the anomeric carbon of the saccharide residue, so as to form an acetal function. Similarly, in formula (V) as defined above, the group R₄ is linked to G₄ via the anomeric carbon of the saccharide residue, so as to form an acetal function.

The term “linear or branched, saturated or unsaturated aliphatic radical including from 12 to 16 carbon atoms” notably denotes for R₃ in formulae (III) and (IV):
a linear alkyl radical chosen from n-dodecyl (n-C₁₂H₂₅—), n-tetradecyl (n-C₁₄H₂₉—) and n-hexadecyl (n-C₁₆H₃₂—) radicals,
a branched alkyl radical derived from the isoalkanols of formula (1):

(CH₃)(CH₃)CH—(CH₂)ᵣ—CH₂—OH (1)

wherein r represents an integer between 8 and 16, for example the isododecyl, isotridecyl, isotetradecyl, isopentadecyl or isohexadecyl radical;
a branched alkyl radical derived from a Guerbet alcohol of formula (2):

CH(CₛH₂ₛ₊₁)(CₜH₂ₜ₊₁)—CH₂—OH (2)

wherein t is an integer between 2 and 12, s is an integer between 2 and 14 and the sum s+t is greater than or equal to 10 and less than or equal to 14, for example the 2-ethyldecyl, 2-butyloctyl, 2-ethyldodecyl, 2-butyldecyl, 2-hexyloctyl, 2-hexyldecyl or 2-butyldodecyl radical.

The compounds of formula (I) as described previously are generally obtained by N-acylation of the corresponding amino acids or of salts thereof. It is described, for example, in the international patent application published under the number WO 98/09611. It is performed equivalently on an amino acid or on an amino acid mixture. The acylating agent generally consists of an activated derivative of the carboxylic acid of formula:

R₁—C(═O)—OH,

wherein R₁ is as defined previously, such as a symmetrical anhydride of this acid, the methyl ester of this acid, or an acid halide such as the acid chloride or the acid bromide. It may also consist of a mixture of activated derivatives of carboxylic acids obtained from natural oils or fats of animal or plant origin such as coconut kernel oil, coconut oil, palm kernel oil, palm oil, soybean oil, rapeseed oil, corn oil, beef tallow, spermaceti oil or herring oil.

According to another particular aspect, the invention relates to the disinfecting composition (C_{D}) according to the invention, characterized in that, wherein said composition (C₁) included in the surfactant mixture (M₁), said composition (C₁) is obtained by a process comprising at least:
a step A) of acylation of a compound of formula (VII):

NH₂—CH(COOH)—(CH₂)₂—COOH (VII),

in acid, partially salified or totally salified acid form, with a mixture of acid chlorides 20 comprising, per 100 mol %, from 40 mol % to 60 mol % of dodecanoyl chloride, from 10 mol % to 20% of tetradecanoyl chloride, from 5 mol % to 15 mol % of decanoyl chloride and from 5 mol % to 15 mol % of octanoyl chloride, and optionally and up to a maximum of 100 mol %, of hexadecanoyl chloride and/or of octadecanoyl chloride and/or of 9-octadecenoyl chloride and/or of octadeca-9,12-dienoyl chloride.

According to a more particular aspect, the mixture of acid chlorides used comprises, per 100 mol %, 11 mol % of octanoyl chloride, 9.5 mol % of decanoyl chloride, 51 mol % of dodecanoyl chloride, 15.5 mol % of tetradecanoyl chloride, 6.5 mol % of hexadecanoyl chloride, 2 mol % of octadecanoyl chloride, 3 mol % of 9-octadecenoyl chloride and 1.5 mol % of octadeca-9,12-dienoyl chloride.

According to another particular aspect, a subject of the invention is the composition (C_{D}) for topical use as defined above, characterized in that, in the composition (C₁) included in the surfactant mixture (M), the compound(s) of formula (I) are chosen from monosodium N-cocoyl glutamate, monopotassium N-cocoyl glutamate, disodium N-cocoyl glutamate and dipotassium N-cocoyl glutamate.

According to an even more particular aspect, a subject of the invention is said composition (C_{D}) for topical use such as defined above, characterized in that the composition (C₃) is obtained by implementing a process comprising at least one step A′) of glycosylation of:
one to five molar equivalents, more particularly from two to four molar equivalents, even more particularly from three to four molar equivalents, of a mixture of alcohols comprising, per 100 mol %:
   from 40 mol % to 90 mol % of 1-dodecanol, more particularly from 65 mol % to 90 mol % and even more particularly from 75 mol % to 90 mol %,
   from 9 mol % to 40 mol % of 1-tetradecanol, more particularly from 9 mol % to 20 mol % and even more particularly from 9 mol % to 20 mol %, and
   from 1 mol % to 20 mol % of 1-hexadecanol, more particularly from 1 mol % to 15 mol % and even more particularly from 1 mol % to 5 mol %,
   with one molar equivalent of a reducing sugar of formula (VII):

HO-(G₃)-H (VII)

wherein G₃ represents a reducing sugar residue chosen from glucose, xylose and arabinose residues.

According to another particular aspect, a subject of the invention is said composition (C_{D}) for topical use as defined above, characterized in that the composition (C₄) is obtained by implementing a process comprising at least one step A₁′ of glycosylation of:
one to four molar equivalents, more particularly from one to three molar equivalents, even more particularly from two to three molar equivalents of at least one alcohol of formula (VI) with 1 molar equivalent of a reducing sugar of formula (VIII):

HO-(G₄)-H (VIII)

wherein G₄ represents a reducing sugar residue chosen from glucose, xylose and arabinose residues.

According to another particular aspect, a subject of the invention is said composition (C_{D}) for topical use as defined above, characterized in that the composition (C₃) is obtained by performing a process comprising at least one step A′ of glycosylation generally performed with mechanical stirring, by bringing 1 molar equivalent of a reducing sugar (G₃) into contact with from 1 to 5 molar equivalents of the mixture of 1-dodecanol, 1-tetradecanol and 1-hexadecanol as described previously, in the presence of an acidic catalytic system, under predetermined temperature and partial vacuum conditions.

Similarly, step A₁′ of the process for preparing composition (C₄), as defined previously, is generally performed with mechanical stirring, by bringing 1 molar equivalent of a reducing sugar (G₄) into contact with from 1 to 4 molar equivalents of at least one alcohol of formula (V), in the presence of an acidic catalytic system, under predetermined temperature and partial vacuum conditions.

Such temperature and partial vacuum conditions are, for example, temperature values of between 70° C. and 130° C. and a partial vacuum of between 300 mbar (3×10⁴ Pa) and 20 mbar (2×10³ Pa). The implementation of step A′ and of step A₁′ of glycosylation makes it possible to form, respectively, composition (C₃), i.e. a mixture of compounds represented by the formulae (III₁), (III₂), (III₃), (III₄) and (III₅) as defined previously, and optionally of an excess of the alcohol of formula (IV) or of the mixture of alcohols of formula (IV), and composition (C₄) i.e. a mixture of compounds represented by the formulae (V₁), (V₂), (V₃), (V₄) and (V₅) as defined previously, and optionally of an excess of the alcohol of formula (VI).

If necessary or if desired, step A′ or step A₁′ of the process for preparing the respective compositions (C₃) and (C₄) as defined previously may be followed, respectively, by a step B′ or a step B₁′ of removal of the alcohols, respectively, of formula (IV), or of the mixture of alcohols of formula (IV), and of formula (VI), which have not reacted during step A′) or step A₁′.

Such a preparation process may be completed, if necessary or if desired, by neutralization, filtration and decolorization operations.

The term “acidic catalytic system” denotes, in step A′ and in step A₁′ of the process defined above, strong acids such as sulfuric acid, hydrochloric acid, phosphoric acid, nitric acid, methanesulfonic acid, para-toluenesulfonic acid, trifluoromethanesulfonic acid, hypophosphorous acid, hyponitrous acid, polyphosphoric acid, or ion-exchange resins.

During step B′) or step B′1) of the process as described above, the alcohols, respectively, of formula (IV), or the mixture of alcohols of formula (IV) or the alcohol of formula (V) are removed according to methods known to those skilled in the art, for instance by distillation, such as thin-film distillation, molecular distillation or solvent extraction.

In the context of the present invention, the term “thickener present in the composition (C_{D}) for topical use that is a subject of the present invention” denotes a chemical compound or a chemical composition which increases the viscosity of the medium into which it is introduced.

In the context of the present invention, the term “gelling agent present in the composition (C_{D}) for topical use that is a subject of the present invention” denotes a chemical compound or a chemical composition which transforms a liquid medium into a structured state, which does not flow, by formation of a three-dimensional network within the liquid; the gel being considered as an intermediate state between the liquid state and the solid state.

In the context of the present invention, the term “polysaccharides” denotes saccharide polymers. The IUPAC definition of saccharides designates monosaccharides, compounds of monosaccharides per se and derivatives thereof, obtained either by reduction of a carbonyl group, or by oxidation of one or more hydroxyl functions, or by the replacement of one or more hydroxyl functions with a hydrogen atom, an amine group, a phosphate function, or a sulfate function. The polysaccharides most commonly used for preparing industrial, food, cosmetic or pharmaceutical compositions predominantly consist of monosaccharides, such as glucose, galactose, mannose or of monosaccharide derivatives for which the hydroxyl function of the terminal carbon has been oxidized to a carboxyl function. Two distinct groups may be distinguished among the polysaccharides: polysaccharides consisting solely of monosaccharides (or poly-monosaccharides) and polysaccharides consisting of monosaccharide derivatives.

According to a particular aspect, the gelling agents and/or thickeners present in the composition (C_{D}) for topical use that is a subject of the present invention are chosen from polysaccharides consisting solely of monosaccharides (or poly-monosaccharides).

Among the polysaccharides composed solely of monosaccharides, a distinction may be made between glucans, which are homopolymers of glucose that are very abundant in nature, glucomannoglycans, xyloglycans and galactomannans, which are polymers of which the main chain consists of D-mannose units, connected together at β-1,4, and on which D-galactose units are grafted laterally by α-1,6 bonds. Galactomannans are present in several plant species, and more particularly in the leguminous species in which they constitute the albumen of seeds. Depending on their plant origin, the degree of substitution (DS) of the D-galactose units on the D-mannose main chain of galactomannans ranges between 0 and 1:
galactomannans originating from cassia gum have a degree of substitution (DS) of approximately ⅕, meaning the lateral grafting of one D-galactose unit every 5 D-mannose units present on the main chain of the polysaccharide;
galactomannans originating from locust bean gum have a degree of substitution (DS) of approximately ¼, meaning the lateral grafting of one D-galactose unit every 4 D-mannose units present on the main chain of the polysaccharide;
galactomannans originating from tara gum have a degree of substitution (DS) of approximately ⅓, meaning the lateral grafting of one D-galactose unit every 3 D-mannose units present on the main chain of the polysaccharide;
galactomannans originating from guar gum have a degree of substitution (DS) of approximately ½, meaning the lateral grafting of one D-galactose unit every 2 D-mannose units present on the main chain of the polysaccharide;
galactomannans originating from fenugreek gum have a degree of substitution (DS) of approximately 1/1, meaning the lateral grafting of one D-galactose unit for virtually every D-mannose unit present on the main chain of the polysaccharide.

According to a more particular aspect, the gelling agents and/or thickeners present in the composition (C_{D}) for topical use that is a subject of the present invention are chosen from polysaccharides consisting solely of monosaccharides (or poly-monosaccharides) included in the group consisting of galactomannan originating from tara gum, galactomannan originating from guar gum and galactomannan originating from locust bean gum.

According to another particular aspect, the gelling agents and/or thickeners present in the composition (C_{D}) for topical use that is a subject of the present invention are chosen from polysaccharides consisting of monosaccharide derivatives. Among the polysaccharides consisting of monosaccharide derivatives, a distinction may be made between:
sulfated galactans, which are polymers of galactose which may have pendant sulfate-ester groups, represented notably by algal polysaccharides such as carrageenans and agar;
uronans, which are the polymers of uronic acids such as algins and pectins;
heteropolymers of monosaccharides and uronic acids: often of complex composition, these polymers are found notably in sap exudates (for instance gum arabic exudate and karaya gum exudate), but they are also produced by microorganisms, for instance xanthan gum and gellan gum;
glucosaminoglycans which are polysaccharides formed from a glucose derived by replacing its C-2 hydroxyl with an amine (referred to as 2-amino-2-deoxy-D-glucose or, more simply, glucosamine). The amine function may also be acetylated. Among the hydrocolloids in this class are chitosan, formed solely of glucosamine units, and hyaluronan, the repeating unit of which is a dimer of glucosamine and glucuronic acid.

Xanthan gum (G_{X}) has in recent decades become the microbial polysaccharide that is the most widely used in industry. Xanthan is a polysaccharide synthesized by bacteria of the genus Xanthomonas and, commercially, only the species X. campestris is used. The main chain of (G_{X}) is identical to that of cellulose, i.e. it is formed from β-D-glucose units connected together via carbons 1 and 4. There is one branched triholoside every two glucose units in the main chain, in a regular alternating manner; each branch consisting of a triholoside composed of two mannoses and a glucuronic acid, of the type: β-D-Manp-(1→4)-β-D-GlcAp-(1→2)-α-D-Manp-(1→3) [I. Capron et al., “About the native and renaturated conformation of xanthan exopolysaccharide” 1997). Xanthan gum (G_{X}) is available in the form of a sodium, potassium or calcium salt.

Acacia gum is a complex, branched polysaccharide the main chain of which consists of β-D-galactose units connected together via carbons 1 and 3. The chains branched to the main chain consist of β-D-galactose units connected together via carbons 1 and 6, also bearing α-arabinose units, and to a lesser extent β-glucoronosyl units. Both the main chain and the pendant chains contain α-L-arabinosyl, α-L-rhamnopyranosyl, β-D-glucuronopyranosyl and 4-O-methyl-α-D-glucuronopyranosyl units.

According to a more particular aspect, the gelling agents and/or thickeners present in the composition (C_{D}) for topical use that is a subject of the present invention are polysaccharides consisting of monosaccharide derivatives chosen from the elements of the group consisting of carrageenans, agar, algins, pectins, gum arabic exudate, karaya gum exudate, xanthan gum, gellan gum, chitosan and hyaluronan, and/or mixtures thereof.

According to another more particular aspect, the gelling agents and/or thickeners present in the composition (C_{D}) for topical use that is a subject of the present invention are polysaccharides consisting of monosaccharide derivatives chosen from the elements of the group consisting of acacia gum exudate, karaya gum exudate and xanthan gum, and/or mixtures thereof.

According to an even more particular aspect, the gelling agents and/or thickeners present in the composition (C_{D}) for topical use that is a subject of the present invention are polysaccharides consisting of monosaccharide derivatives chosen from the elements of the group consisting of acacia gum exudate (G_{A}), xanthan gum (G_{X}), the mixture of xanthan gum (G_{X}) and acacia gum exudate (G_{A}) used in a mass ratio between the xanthan gum (G_{X}) and the acacia gum exudate (G_{A}) of greater than or equal to ⅓ and less than or equal to 3/1, sold notably by the company SEPPIC under the brand name Solagum™ AX.

According to one particular aspect, the gelling and/or thickening agents present in the composition (C_{D}) for topical use that is the subject of the present invention are chosen from cellulose and cellulose derivatives.

In the context of the present invention, the term “cellulose” denotes a polysaccharide consisting of a linear chain of D-glucose molecules, the average molecular mass of which is at least 10 000 g·mol⁻¹, more particularly at least 15 000 g·mol⁻¹, more particularly at least 17 000 g·mol⁻¹, even more particularly at least 20 000 g·mol⁻¹ and even more particularly at least 25 000 g·mol⁻¹.

According to a more particular aspect, the gelling agents and/or thickeners present in the composition (C_{D}) for topical use that is a subject of the present invention are chosen from cellulose derivatives.

In the context of the present invention, the term “cellulose derivatives” denotes the elements of the group consisting of hydroxyethylcellulose, methylcellulose, ethylcellulose, methylhydroxyethylcellulose, methylhydroxypropylcellulose, hydroxypropylcellulose, the sodium salt of carboxymethylcellulose, and cellulose dihydroxypropyl ether (as described in the American patent published under the number U.S. Pat. No. 4,096,326).

In the context of the present invention, the term “starch” denotes a mixture of amylose and amylopectin, and more particularly the elements of the group consisting of corn starch, wheat starch, potato starch and cassava starch.

According to a particular aspect, the term “linear or branched or crosslinked polymers of polyelectrolyte type” denotes, for the purposes of the present invention:
crosslinked synthetic anionic copolymers based on methacrylic acid or acrylic acid, or esters of methacrylic acid or of acrylic acid, which are optionally hydrophically modified, prepared by direct emulsion polymerization. These synthetic anionic copolymers are known, respectively, to those skilled in the art under the names “Alkaline Swellable Emulsion” (or “ASE”) and “Hydrophobically Alkaline Swellable Emulsion” (or “HASE”). Thickeners of HASE type are described in the international patent application published under the number WO 02/34793 A2;
crosslinked or branched synthetic anionic polyelectrolytes, which are crosslinked and/or branched homopolymers or copolymers of water-soluble unsaturated monomers, such as acrylic acid and/or derivatives thereof, methacrylic acid and/or derivatives thereof, acrylamide and/or derivatives thereof, 2-acrylamido-2-methylpropanesulfonic acid and/or salts thereof, N-vinylpyrrolidone, vinyl alcohol and/or derivatives thereof. These crosslinked or branched synthetic anionic polyelectrolytes are in the form of reverse latices, obtained by reverse emulsion radical polymerization, or in the form of powders, obtained by precipitating polymerization, or by atomization of reverse latices.

According to a particular aspect, the gelling agents and/or thickeners present in the composition (C_{D}) for topical use that is a subject of the present invention are chosen from linear or branched or crosslinked polyelectrolytes, obtained from the radical polymerization of at least one monomer chosen from the elements of the group consisting of acrylic acid and/or the sodium salt thereof, methacrylic acid and/or the sodium salt thereof, 2-hydroxyethyl acrylate, 2-hydroxyethyl methacrylate, acrylamide, N,N-dimethylacrylamide, N-isopropylacrylamide, 2-acrylamido-2-methylpropanesulfonic acid and/or the sodium or the potassium salt thereof, N-vinylpyrrolidone, in the presence of a crosslinking agent chosen from polyethylenic monomers comprising at least two ethylenic functions, and more particularly chosen from elements of the group consisting of ethylene glycol dimethacrylate, tetraallyloxyethane, ethylene glycol diacrylate, diallylurea, triallylamine, trimethylolpropane triacrylate or methylenebis(acrylamide), or a mixture of these compounds.

According to a particular aspect, the gelling agents and/or thickeners present in the composition (C_{D}) for topical use that is a subject of the present invention are chosen from the elements of the group consisting of:
partially or totally salified acrylic acid homopolymer, crosslinked with triallylamine and/or with trimethylolpropane triacrylate and/or with methylenebis(acrylamide),
the homopolymer of the sodium salt of 2-acrylamido-2-methylpropanesulfonic acid, crosslinked with triallylamine and/or with trimethylolpropane triacrylate and/or with methylenebis(acrylamide),
the copolymer of the sodium salt of 2-acrylamido-2-methylpropanesulfonic acid and of partially or totally salified acrylic acid, crosslinked with triallylamine and/or with trimethylolpropane triacrylate and/or with methylenebis(acrylamide),
the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide),
the copolymer of the sodium salt of 2-acrylamido-2-methylpropanesulfonic acid and of acrylamide, crosslinked with triallylamine and/or with trimethylolpropane triacrylate and/or with methylenebis(acrylamide),
the terpolymer of the sodium salt of 2-acrylamido-2-methylpropanesulfonic acid, of acrylamide and of partially or totally salified acrylic acid, crosslinked with triallylamine and/or with trimethylolpropane triacrylate and/or with methylenebis(acrylamide),
the terpolymer of the sodium salt of 2-acrylamido-2-methylpropanesulfonic acid, of N,N-dimethylacrylamide and of partially or totally salified acrylic acid, crosslinked with triallylamine and/or with trimethylolpropane triacrylate and/or with methylenebis(acrylamide).

According to a more particular aspect, the gelling agents and/or thickeners present in the composition (C_{D}) for topical use that is a subject of the present invention are chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to ⅓ and less than or equal to 3/1, the copolymer of the sodium salt of 2-acrylamido-2-methylpropanesulfonic acid and of 2-hydroxyethyl acrylate, crosslinked with triallylamine and/or with trimethylolpropane triacrylate and/or with methylenebis(acrylamide), the copolymer of the sodium salt of 2-acrylamido-2-methylpropanesulfonic acid and of acrylamide, crosslinked with triallylamine and/or with trimethylolpropane triacrylate and/or with methylenebis(acrylamide).

In the context of the present invention, the term “disinfectant” denotes a chemical substance or composition which kills or inactivates microorganisms present on the surface of the skin to which it has been applied.

The disinfectants included in the composition (C_{D}) for topical use that is a subject of the present invention belong to biocidal products as defined by the regulations concerning the making available on the market and use of biocidal products (EU Regulation No. 528/2012 of May 22, 2012).

Biocidal products represent all substances and mixtures, consisting of one or more active molecules, intended to destroy, repel or render harmless living pest organisms, to prevent their action or to combat them in any other way by chemical or biological action. These products are divided, depending on their applications, into four groups which are (i) disinfectants, (ii) protective products aimed at preventing microbial growth and algal growth, (iii) pest control products and (iv) other biocidal products such as anti-fouling products or for embalming and taxidermy.

The effectiveness of disinfectants depends on their spectrum of action on the different types of biological agents. Bactericidal agents (action on bacteria), fungicidal agents (action on fungi), virucidal agents (action on viruses) and sporicidal agents (action on spores) are defined in this way. In addition, each disinfectant has several performance criteria, such as (i′) its speed of efficacy, (ii′) its decontamination efficacy which is measured by a factor of reduction of an initial contaminating population under the effect of the disinfectant (initial population/final population after treatment) or by the log reduction of this factor, and (iii′) its compatibility with construction materials. Disinfectants are therefore classified according to their disinfection efficacy and references are made to disinfectants with a high, medium or low level of disinfection.

The composition (C_{D}) for topical use that is a subject of the present invention comprises at least one disinfectant chosen from disinfectants with a high level of disinfection, namely disinfectants exhibiting a factor (initial contaminating population/final population after treatment) of greater than 10⁶.

According to one particular aspect, a subject of the invention is a composition (C_{D}) for topical use as defined previously, characterized in that the at least disinfectant is chosen from the elements of the group consisting of sorbic acid, sodium sorbate, potassium sorbate, dehydroacetic acid, sodium dehydroacetate, benzoic acid, sodium benzoate, potassium benzoate; 1-(2-ethylhexyl) glycerol; hydrogen peroxide; activated peroxides such as mixtures of hydrogen peroxide and sodium bicarbonate, mixtures of hydrogen peroxide and urea, mixtures of hydrogen peroxide and peracetic acid and mixtures of hydrogen peroxide and iron (Fenton's reagent); hydroperoxycarbonates; peracetic acid; sodium hypochlorite.

According to one particular aspect, a subject of the invention is a composition (C_{D}) for topical use as defined previously, characterized in that the at least disinfectant is hydrogen peroxide or sodium hypochlorite.

According to another particular aspect, a subject of the invention is a composition (C_{D}) for topical use as defined previously, characterized in that the at least disinfectant is a mixture of potassium sorbate and sodium benzoate, and even more particularly a mixture comprising, per 100% of its mass, (i) from 30% to 40% by mass of potassium sorbate and from 60% to 70% by mass of sodium benzoate.

According to another particular aspect, a subject of the invention is a composition (C_{D}) for topical use as defined previously, characterized in that the at least disinfectant is 1-(2-ethylhexyl) glycerol.

According to another particular aspect, a subject of the invention is a composition (C_{D}) for topical use as defined previously, characterized in that the at least disinfectant is a mixture of benzyl alcohol, benzoic acid and dehydroacetic acid.

The composition (C_{D}) for topical use that is a subject of the present invention can be packaged in pressurized form in an aerosol device or in a device of “pump bottle” type. When it is packaged in bottles, the composition (C_{D}) for topical use according to the invention as defined previously may also be applied in the form of fine droplets by means of mechanical pressurization devices or via propellant gas devices. Among the propellants that may be combined with the composition (C_{D}) for topical use according to the invention are hydrofluoro compounds, for instance dichlorodifluoromethane, trichlorofluoromethane, difluoroethane, isobutane, butane and propane.

The composition (C_{D}) for topical use as defined previously may also include excipients and/or active agents commonly used in the field of formulations for topical use, in particular cosmetic, dermocosmetic, pharmaceutical or dermopharmaceutical formulations.

The composition (C_{D}) for topical use, which is the subject of the present invention and as defined previously, may also comprise one or more auxiliary compounds chosen from thickening and/or gelling surfactants, film-forming compounds, solvents and cosolvents, hydrotropic agents, plasticizers, opacifiers, nacreous agents, sequestrants, chelating agents, antioxidants, fragrances, essential oils, preserving agents, conditioning agents, deodorants, bleaching agents intended for bleaching bodily hair and the skin, active ingredients intended to provide a treating and/or protective action to the skin or the hair, sunscreens, mineral fillers or pigments, particles that give a visual effect or that are intended for encapsulating active agents, exfoliant particles, texture agents, optical brighteners and insect repellents.

Among the water-soluble antioxidants that may be combined with the composition (C_{D}) for topical use according to the invention are ascorbic acid, glutathione, tartaric acid, oxalic acid and tetrasodium glutamate diacetate.

Among the water-soluble sequestering agents which can be combined with the composition (C_{D}) for topical use according to the invention are salts of ethylenediamine tetracetic acid (EDTA), such as the sodium salt of EDTA, salts of diethylenetriamine pentacetic acid (DTPA) such as sodium salts of DTPA, acetyl glutamic acid (Dissolvine range).

Among the water-soluble dyes that may be combined with the composition (C_{D}) for topical use according to the invention are caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3, Yellow 10.

Among the water-soluble color stabilizing agents which can be combined with the composition (C_{D}) for topical use according to the invention, there is Tris citrate (tetramethyl hydroxy piperidinol), sodium benzotriazolyl butylphenol sulfonate, benzotriazolyl dodecyl p-cresol.

As examples of texture agents optionally present in the composition (C_{D}) for topical use which is a subject of the present invention, mention may be made of N-acylamino acid derivatives, for example lauroyl lysine sold under the name Aminohope™LL, octenyl starch succinate sold under the name Dryflo™, myristyl polyglucoside sold under the name Montanov™ 14, cellulose fibers, cotton fibers, chitosan fibers, talc, sericite, mica and perlite.

Examples of active ingredients optionally present in the composition (C_{D}) for topical use that is a subject of the present invention include:
vitamins and derivatives thereof, for example retinol (vitamin A) and esters thereof (for example retinyl palmitate), ascorbic acid (vitamin C) in salt form and esters thereof, sugar derivatives of ascorbic acid (for example ascorbyl glucoside), tocopherol (vitamin E) and esters thereof (for example tocopheryl acetate), vitamin B3 or B10 (niacinamide and derivatives thereof); compounds having a lightening or depigmenting action on the skin, for example Sepiwhite™ MSH, arbutin, kojic acid, hydroquinone, Vegewhite™, Gatuline™, Synerlight™ Biowhite™, Phytolight™, Dermalight™, Clariskin™, Melaslow™, Dermawhite™, Ethioline, Melarest™, Gigawhite™, Albatine™ and Lumiskin™;
compounds with a calmative action, such as Sepicalm™ S, allantoin and bisabolol;
anti-inflammatory agents;
compounds with moisturizing action, for example diglycerol, triglycerol, urea, hydroxyureas, glycerol glucoside, diglycerol glucoside, polyglyceryl glucosides, erythrityl glucoside, sorbityl glucoside, xylityl glucoside, the composition sold under the brand name Aquaxyl™ comprising xylityl glucoside, anhydroxylitol and xylitol;
compounds with slimming or lipolytic action, such as caffeine or derivatives thereof, Adiposlim™ and Adipoless™;
plant extracts rich in tannins, polyphenols and/or isoflavones, for example grape extracts, pine extracts, wine extracts, olive extracts; soybean extracts, for example Raffermine™; wheat extracts, for example Tensine™ or Gliadine™; terpene-rich plant extracts; freshwater or seawater algal extracts; marine extracts in general such as corals;
compounds with antimicrobial action or with purifying action, for example Lipacide™ C8G, Lipacide™ UG, Sepicontrol™ A5; Octopirox™ or Sensiva™ SC50;
compounds with an energizing or stimulating property, such as Physiogenyl™ panthenol and derivatives thereof such as Sepicap™ MP;
antiaging active agents such as Sepilift™ DPHP, Lipacide™ PVB, Sepivinol™ Sepivital™, Manoliva™, Phyto-Age™, Timecode™ or Survicode™;
active agents for combating photoaging;
active agents which increase the synthesis of extracellular matrix components, for example collagen, elastins and glycosaminoglycans;
active agents acting favorably on chemical cellular communication, such as cytokines, or on physical cellular communication, such as integrins;
active agents which create a “heating” sensation on the skin, such as skin capillary circulation activators (for example nicotinic acid derivatives) or products which create a “freshness” sensation on the skin (for example menthol and derivatives thereof);
active agents which improve the skin capillary circulation, for example venotonic agents; draining active agents; decongesting active agents, for example extracts of Ginkgo biloba, ivy, common horse chestnut, bamboo, ruscus, butcher's broom, Centella asiatica, fucus, rosemary or willow;
active agents acting as skin-tautening agents, for example plant protein hydrolyzates, hydrolyzates of marine origin, for instance hydrolyzates of laminaria extracts, fish cartilage hydrolyzates, marine elastin, the product sold by the company SEPPIC under the brand name Sesaflash™, and collagen solutions;
skin tanning or browning agents, for example dihydroxyacetone, isatin, alloxan or ninhydrin, glyceraldehyde, mesotartaric aldehyde, glutaraldehyde or erythrulose.

As examples of deodorants optionally present in the composition (C_{D}) for topical use which is a subject of the present invention, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of oils that may be present in the composition (C_{D}) for topical use that is a subject of the present invention, mention may be made of mineral oils such as liquid paraffin, liquid petroleum jelly, isoparaffins or white mineral oils; oils of animal origin such as squalene or squalane; plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, coriander seed oil, beechnut oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(α-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

As examples of waxes that may be present in the composition (C_{D}) for topical use that is a subject of the present invention, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at ambient temperature; glycerides that are solid at ambient temperature. In the present patent application, the term “waxes” refers to compounds and/or mixtures of compounds which are water-insoluble, and which have a solid appearance at a temperature of greater than or equal to 45° C.

As examples of emulsifying nonionic surfactants that may be combined with the composition (C_{D}) for topical use that is a subject of the present invention, mention may be made of sorbitol and fatty acid esters, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucoside esters; alkyl polyglycosides including a linear or branched, saturated or unsaturated aliphatic radical, and including from 14 to 36 carbon atoms, for instance tetradecyl polyglucoside, hexadecyl polyglucoside, octadecyl polyglucoside, hexadecyl polyxyloside, octadecyl polyxyloside, eicosyl polyglucoside, dodecosyl polyglucoside, (2-octyldodecyl) polyxyloside, (12-hydroxystearyl) polyglucoside; compositions of linear or branched, saturated or unsaturated fatty alcohols including from 14 to 36 carbon atoms and of alkyl polyglycosides as described previously, for example the compositions sold under the brand names Montanov™ 68, Montanov™ 14, Montanov™ 82, Montanov™ 202, Montanov™ S, Montanov™ WO18, Montanov™ L, Fluidanov™ 20× and Easynov™.

As examples of agents for protecting against the ultraviolet rays of the sun that may be present in the composition (C_{D}) for topical use that is a subject of the present invention, pigments, organic sunscreens and inorganic sunscreens are denoted.

As pigments used as agents for protecting against the ultraviolet rays of the sun that may be present in the composition (C_{D}) for topical use that is a subject of the present invention, there are, for example, titanium dioxide, brown iron oxides, yellow iron oxides, black iron oxides or red iron oxides, or else white or colored nacreous pigments such as titanium mica.

As organic sunscreens used as agents for protecting against the ultraviolet rays of the sun that may be present in the composition (C_{D}) for topical use that is a subject of the present invention, there are, for example:
those of the family of benzoic acid derivatives, such as para-aminobenzoic acids (PABAs), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA;
those of the family of anthranilic acid derivatives, such as homomenthyl-N-acetyl anthranilate;
those of the family of salicylic acid derivatives, such as amyl salicylate, homomenthyl salicylate, ethylhexyl salicylate, phenyl salicylate, benzyl salicylate, p-isopropanolphenyl salicylate;
those of the family of cinnamic acid derivatives, such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy 2-ethoxyethyl cinnamate, p-methoxycyclohexyl cinnamate, ethyl-α-cyano-β-phenyl cinnamate, 2-ethylhexyl-□α-cyano-β-phenyl cinnamate, glyceryl di-para-methoxy mono-2-ethylhexanoyl cinnamate;
those of the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxy benzophenone-5-sulfonate, 4-phenylbenzophenone, 2-ethylhexyl-4′-phenyl benzophenone-2-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, benzalkonium methosulfate camphor; urocanic acid, ethyl urocanate;
those of the family of sulfonic acid derivatives, such as 2-phenylbenzimidazole-5-sulfonic acid and its salts; the family of triazine derivatives, such as hydroxyphenyl triazine, ethylhexyloxyhydroxyphenyl-4-methoxyphenyltriazine, 2,4,6-trianilino-(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis(2-ethylhexyl) ester of benzoic acid, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris [4-(2-ethylhexyloxycarbonyl)anilino]-1,3,5-triazine, 2-ethylhexyl dimethoxybenzylidene dioxoimidazolidine propionate, the family of diphenylacrylate derivatives, such as 2-ethylhexyl-2-cyano-3,3-diphenyl-2-propenoate or ethyl-2-cyano-3,3-diphenyl-2-propenoate;
those of the family of the polysiloxanes, such as benzylidene siloxane malonate.
   As inorganic sunscreens used as agents for protecting against the ultraviolet rays of the sun that may be present in formulation (F₂) for topical use that is a subject of the present invention, there are, for example, titanium oxides, zinc oxides, cerium oxide, zirconium oxide, yellow, red or black iron oxides, and chromium oxides. These mineral sunblocks may or may not be micronized, may or may not have undergone surface treatments and may optionally be in the form of aqueous or oily predispersions.

A subject of the invention is also a disinfecting composition (C_{D}) according to the invention for disinfectant washing of the hands.

For the purposes of the present invention, the term “disinfectant washing” or “hygienic washing” or “antiseptic washing” of the hands is intended to mean a process which makes it possible to carry out effective and well tolerated disinfection of the hands by washing.

The most frequent applications thereof are found in the hospital or medical environment, the disinfection of the hands taking place immediately before an aseptic procedure or after contact with a patient in isolation, but the disinfectant washing is carried out by any individual who wishes to avoid contamination by micro-organisms present on hard surfaces with which the hands are in contact (door handles, hard surfaces in public transport, etc.).

Disinfectant washing is carried out with an antiseptic soap, the efficacy of which is validated by compliance with European and AFNOR standards.

For the use according to the invention, the composition (C_{D}) for topical use as defined previously is implemented in order to create a foam, generated:
by rubbing the composition (C_{D}) for topical use between both hands, or
by rubbing the composition (C_{D}) for topical use previously deposited on the surface of the skin to be treated,
by using pressurized packaging, which contains the composition (C_{D}) for topical use combined with a propellant, generating the foam which is then applied to the skin area to be treated,
by using pressurized packaging in an aerosol device, without a propellant, emitting fine droplets applied to the skin and then rubbed to generate the foam.

A subject of the invention is also a disinfecting composition (C_{D}) according to the invention for treating a skin infection, a skin burn, a scar, a wound, in human beings or animals.

It should be noted that the wounds and skin burns can be subject to surgical procedures.

### DESCRIPTION OF THE PREFERRED EMBODIMENTS

The examples that follow illustrate the invention without, however, limiting it.

### 1) Preparation of Foaming Compositions

### 1.1) Preparation of a Solution of Disodium N-Cocoyl Glutamate [Composition (C₁)].

375 kg of water and 250 kg of monosodium sodium glutamate monohydrate are placed in a reactor, with stirring and at a temperature of 20° C., followed by 184 kg of an aqueous sodium hydroxide solution at 30% by mass so as to reach a pH of 12.245 kg of cocoyl chloride, which is a mixture of acid chlorides comprising, per 100% by mass, 8% by mass of octanoyl chloride, 8% by mass of decanoyl chloride, 50% by mass of lauroyl chloride, 17% by mass of myristoyl chloride, 8% by mass of palmitoyl chloride, 3% by mass of stearoyl chloride, 4% by mass of oleoyl chloride and 2% by mass of linoleoyl chloride, are then added gradually with stirring, followed by a further 140 kg of the 30% sodium hydroxide solution to keep the pH between 11 and 12. The temperature is maintained between 20° C. and 50° C. for 2 hours.

The mixture obtained is acidified by adding 54 kg of an aqueous sulfuric acid solution at 70% by mass, and is then diluted with 193 kg of water to obtain an aqueous solution of disodium N-cocoyl glutamate [composition (C₁)].

### 1.2) Analytical Features of the Solutions Prepared Previously

The analytical features of composition (C₁) are collated in table 1 below.

TABLE 1

| | (C₁) |
|---|---|
| Appearance at 20° C. (visual method) | Clear |
| Mass content of water (a) (according to the standard | 68.00% |
| NFT 73-201) | |
| Residual fatty acids (b) (gas chromatography (GC)) | 5.8% |
| pH | 6.3 |
| Sodium chloride content (potentiometric titration (c)) | 3.60% |
| Sodium sulfate content (d) (calculated on feedstock) | 5.20% |
| Citrate content (e) (calculated on feedstock) | 0% |
| Mass content of active material (AM₁) (cocoyl glutamate) | 17.40% |
| (AM₁) = 100% − (a) − (b) − (c) − (d) − (e) | |

### 2) Preparation of the Alkyl Polyglycoside-Based Surfactant Compositions

### 2.1) Preparation of a Composition (C₃)

3.7 molar equivalents of a mixture of fatty alcohols (N₁) consisting, per 100% of its mass, of 68% by mass of 1-dodecanol, of 25% by mass of 1-tetradecanol and of 7% by mass of 1-hexadecanol, and then 1 molar equivalent of anhydrous glucose are poured with stirring into a reactor maintained at 80° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed.

After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess of the mixture of fatty alcohols (N₁) is removed by distillation using a thin-film evaporator, and the residue is then diluted in water. After stirring for 30 minutes at 50° C., composition (C₃) is obtained, which comprises 50% by mass of water and 50% by mass of a mixture of alkyl polyglucosides (AM_{APG1}), for which the proportions of alkyl polyglucosides and the mean degree of polymerization of their polyglucoside residue are determined by gas chromatography (GC); it thus comprises, per 100% by mass, 69% by mass of n-dodecyl polyglucosides, 25% by mass of n-tetradecyl polyglucosides and 6% by mass of n-hexacyl polyglucosides with a degree of polymerization equal to 1.25.

2.2) Preparation of a Composition (C₄) Comprising n-Heptyl Polyglucoside

2.7 molar equivalents of 1-heptanol and then 1 molar equivalent of anhydrous glucose are poured with stirring into a reactor maintained at 40° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed. After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess heptanol is distilled off under partial vacuum, and the residue is then diluted in water.

After stirring for 30 minutes at 50° C., composition (C₄) is obtained comprising 26.4% by mass of water and 73.6% by mass of n-heptyl polyglucosides (AM_{APG2}), with a degree of polymerization, determined by GC, equal to 1.25.

2.3) Preparation of a Composition (C₅) Comprising n-Octyl Polyglucoside and n-Decyl Polyglucoside

2.7 molar equivalents of a mixture of fatty alcohols (N₃) consisting, per 100% of its mass, of 50% by mass of 1-octanol and of 50% by mass of 1-decanol, and then 1 molar equivalent of anhydrous glucose are poured with stirring into a reactor maintained at 80° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed.

After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess of the mixture of fatty alcohols (N₃) is removed by distillation using a thin-film evaporator, and the residue is then diluted in water.

After stirring for 30 minutes at 50° C., composition (C) is obtained, which comprises 40% by mass of water and 60% by mass of a mixture of alkyl polyglucosides (AM_{APG3}), for which the proportions of alkyl polyglucosides and the mean degree of polymerization of their polyglucoside residue are determined by GC; it thus comprises, per 100% by mass, 52% by mass of n-octyl polyglucoside and 48% by mass of n-decyl polyglucoside, with an average degree of polymerization equal to 1.30.

### 2.4) Preparation of Compositions Based on Disodium Cocoylglutamate and Alkyl Polylglucosides

2.4.1) Preparation of a Composition (T₁) Comprising Disodium N-Cocoylglutamate [Composition (C₁)], a Mixture of n-Dodecyl Polyglucosides, n-Tetradecyl Polyglucosides and n-Hexadecyl Polyglucosides [Composition (C₃)], and n-Heptyl Polyglucoside [Composition (C₄)]

A composition (T₁) is prepared by pouring, with stirring, the composition (C₁) and the compositions (C₃) and (C₄) into a reactor maintained at 40° C. The mixture is stirred for thirty minutes in order to obtain the composition (T₁). The amounts used are listed in table 2 below.

2.4.2) Preparation of a Composition (T₂) Comprising Disodium N-Cocoylglutamate [Composition (C₁)], a Mixture of n-Dodecyl Polyglucosides, n-Tetradecyl Polyglucosides and n-Hexadecyl Polyglucosides [Composition (C₃)]

A composition (T₂) is prepared by pouring, with stirring, the composition (C₁) and the composition (C₃) into a reactor maintained at 40° C. The mixture is stirred for thirty minutes in order to obtain the composition (T₂). The amounts used are listed in table 2 below.

TABLE 2
Amounts used

| | (C₁) | (C₃) | | (C₄) | |
|---|---|---|---|---|---|
| (T₁) | 74.9 g | 11.1 | g | 14.0 | g |
| (T₂) | 93.0 g | 7.0 | g | 0 | g |

The analytical features of the compositions (T₁) and (T₂) are collated in table 3 below.

TABLE 3

| | | | | H₂O | | AMC⁽⁵⁾ | |
|---|---|---|---|---|---|---|---|
| | FA⁽¹⁾ | Δ⁽²⁾ | T′⁽³⁾ | (%) | Δ₁⁽⁴⁾ | as % | Appearance |
| (T₁) | 5.84% | 0.45 | 0.55 | 60.15% | 0.54 | 28.85% | Homogeneous |
| (T₂) | 5.4% | 4.6 | 0.18 | 66.74% | — | 19.68% | Homogeneous |

⁽¹⁾Residual fatty acids (mass percentage)
⁽²⁾Δ = (AM₁)/[(AM_{APG1}) + (AM_{APG2})]
⁽³⁾T′ = [(AM_{APG1}) + (AM_{APG2})]/[(AM₁) + (AM_{APG1}) + (AM_{APG2})]
⁽⁴⁾Δ₁ = (AM_{APG1})/(AM_{APG2})
⁽⁵⁾AMC = [(AM₁) + (AM_{APG1}) + (AM_{APG2})]/total mass, with total mass = 100 g.

### 3) Preparation of Compositions According to the Invention and of Comparative Compositions

3.1) Preparation of Compositions According to the Invention E₁ to E₈ and of a Comparative Composition E₀

The compositions E₀ to E₈ are prepared at a temperature of 25° C., in a reactor of suitable volume fitted with anchor-type mechanical stirring at a speed of 50 revolutions·min⁻¹. The ingredients are gradually introduced one after the other until a homogeneous and liquid composition is obtained.

The compositions are described in detail in table 4 below:

TABLE 4

| | E₀ | E₁ | E₂ | E₃ | E₄ |
|---|---|---|---|---|---|
| Composition | 2.36%, i.e. | 2.36%, i.e. | 2.36%, i.e. | 2.36%, i.e. | 2.36%, i.e. |
| (T₁) at | 0.68% of | 0.68% of | 0.68% of | 0.68% of | 0.68% of |
| 28.85% of | AMC⁽⁵⁾ | AMC⁽⁵⁾ | AMC⁽⁵⁾ | AMC⁽⁵⁾ | AMC⁽⁵⁾ |
| AMC⁽⁵⁾ | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.5 | 6.4 | 6.7 | 6.4 | 6.2 |
| Visual | Homogeneous | Homogeneous | Homogeneous | Homogeneous | Homogeneous |
| appearance | liquid | liquid | liquid | liquid | liquid |
| product | | | | | |

TABLE 4 (a)

| | E₅ | E₆ | E₇ | E₈ |
|---|---|---|---|---|
| Composition | 2.36%, i.e. | 2.36%, i.e. | 2.36%, i.e. | 2.36%, i.e. |
| (T₁) at 28.5% of | 0.68% of | 0.68% of | 0.68% of | 0.68% of |
| AMC⁽⁵⁾ | AMC⁽⁵⁾ | AMC⁽⁵⁾ | AMC⁽⁵⁾ | AMC⁽⁵⁾ |
| Xanthan solution | 15% | 15% | 15% | 15% |
| at 1.66% by mass | | | | |
| Deionized water | q.s. 100% | q.s. 100% | q.s. 100% | q.s. 100% |
| Euxyl ™K712⁽⁶⁾ | 5% | 10% | — | — |
| Euxyl ™K903⁽⁷⁾ | — | — | 2.5% | — |
| Euxyl ™PE9010⁽⁸⁾ | — | — | — | 2.5% |
| pH product | 6.5 | 6.4 | 6.5 | 6.5 |
| Visual | Homogeneous | Homogeneous | Homogeneous | Homogeneous |
| appearance | liquid | liquid | liquid | liquid |
| product | | | | |

⁽⁶⁾Euxyl ™K712: aqueous solution containing 45% of active material, said active material comprising, per 100% of its mass, 33% by mass of potassium sorbate and 67% by mass of sodium benzoate.
⁽⁷⁾Euxyl ™ K903: mixture of benzyl alcohol, benzoic acid, dehydroacetic acid and tocopherol.
⁽⁸⁾Euxyl ™ PE9010: mixture of 2-phenoxyethanol and 1-(2-ethylhexyl) glycerol

3.2) Preparation of Comparative Compositions F₀ to F₄, Comprising the Composition (T₂)

The compositions F₀ to F₄ are prepared according to the procedure described in section 3.1 above.

The compositions F₀ to F₄ are described in detail in table 5 below:

TABLE 5

| | F₀ | F₁ | F₂ | F₃ | F₄ |
|---|---|---|---|---|---|
| Composition | 3.45%, i.e. | 3.45%, i.e. | 3.45%, i.e. | 3.45%, i.e. | 3.45%, i.e. |
| (T₂) at | 0.68% of | 0.68% of | 0.68% of | 0.68% of | 0.68% of |
| 19.68% of | AMC⁽⁵⁾ | AMC⁽⁵⁾ | AMC⁽⁵⁾ | AMC⁽⁵⁾ | AMC⁽⁵⁾ |
| AMC⁽⁵⁾ | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.4 | 6.4 | 6.5 | 6.2 | 6.0 |
| Visual | Homogeneous | Homogeneous | Homogeneous | Homogeneous | Homogeneous |
| appearance | liquid | liquid | liquid | liquid | liquid |
| product | | | | | |

3.3) Preparation of Comparative Compositions F₅ to F₉, Comprising the Composition (C₁)

The compositions F₅ to F₉ are prepared according to the procedure described in section 3.1 above.

The compositions F₅ to F₉ are described in detail in table 6 below:

TABLE 6

| | F₅ | F₆ | F₇ | F₈ | F₉ |
|---|---|---|---|---|---|
| Composition | 3.90%, i.e. | 3.90%, i.e. | 3.90%, i.e. | 3.90%, i.e. | 3.90%, i.e. |
| (C₁) at | 0.68% of | 0.68% of | 0.68% of | 0.68% of | 0.68% of |
| 17.4% of | AM1 | AM1 | AM1 | AM1 | AM1 |
| AM1 | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.3 | 6.8 | 6.7 | 6.5 | 6.2 |
| Visual | Homogeneous | Homogeneous | Homogeneous | Homogeneous | Homogeneous |
| appearance | liquid | liquid | liquid | liquid | liquid |
| product | | | | | |

3.4) Preparation of Comparative Compositions F₁₀ to F₁₄, Comprising the Composition (C₃)

The compositions F₁₀ to F₁₄ are prepared according to the procedure described in section 3.1 above.

The compositions F₁₀ to F₁₄ are described in detail in table 7 below:

TABLE 7

| | F₁₀ | F₁₁ | F₁₂ | F₁₃ | F₁₄ |
|---|---|---|---|---|---|
| Composition | 1.36%, i.e. | 1.36%, i.e. | 1.36%, i.e. | 1.36%, i.e. | 1.36%, i.e. |
| (C₃) at | 0.68% of | 0.68% of | 0.68% of | 0.68% of | 0.68% of |
| 50.0% of | AM_{APG1} | AM_{APG1} | AM_{APG1} | AM_{APG1} | AM_{APG1} |
| AM_{APG1} | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.4 | 6.6 | 6.8 | 6.9 | 6.1 |
| Visual | Homogeneous | Homogeneous | Homogeneous | Homogeneous | Homogeneous |
| appearance | liquid | liquid | liquid | liquid | liquid |
| product | | | | | |

3.5) Preparation of Comparative Compositions F₁₅ to F₁₉, Comprising the Composition (C₄)

The compositions F₁₅ to F₁₉ are prepared according to the procedure described in section 3.1 above.

The compositions F₁₅ to F₁₉ are described in detail in table 8 below:

TABLE 8

| | F₁₅ | F₁₆ | F₁₇ | F₁₈ | F₁₉ |
|---|---|---|---|---|---|
| Composition | 0.92%, i.e. | 0.92%, i.e. | 0.92%, i.e. | 0.92%, i.e. | 0.92%, i.e. |
| (C₄) at | 0.68% of | 0.68% of | 0.68% of | 0.68% of | 0.68% of |
| 73.6% of | AM_{APG2} | AM_{APG2} | AM_{APG2} | AM_{APG2} | AM_{APG2} |
| AM_{APG2} | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.3 | 6.5 | 6.5 | 6.4 | 6.5 |
| Visual | Homogeneous | Homogeneous | Homogeneous | Homogeneous | Homogeneous |
| appearance | liquid | liquid | liquid | liquid | liquid |
| product | | | | | |

3.6) Preparation of Comparative Compositions F₂₀ to F₂₄, Comprising the Composition (C₄)

The compositions F₂₀ to F₂₄ are prepared according to the procedure described in section 3.1 above.

The compositions F₂₀ to F₂₄ are described in detail in table 9 below:

TABLE 9

| | F₂₀ | F₂₁ | F₂₂ | F₂₃ | F₂₄ |
|---|---|---|---|---|---|
| Composition | 1.13%, i.e. | 1.13%, i.e. | 1.13%, i.e. | 1.13%, i.e. | 1.13%, i.e. |
| (C₅) at | 0.68% of | 0.68% of | 0.68% of | 0.68% of | 0.68% of |
| 60.0% of | AM_{APG3} | AM_{APG3} | AM_{APG3} | AM_{APG3} | AM_{APG3} |
| AM_{APG3} | | | | | |
| Xanthan | 15% | 15% | 15% | 15% | 15% |
| solution at | | | | | |
| 1.66% by | | | | | |
| mass | | | | | |
| Deionized | q.s. 100% | q.s. 100% | q.s. 100% | q.s. 100% | q.s. 100% |
| water | | | | | |
| Bleach at | — | 7.1% by | 21.4% by | — | — |
| 12° | | mass of | mass of | | |
| chlorine | | NaOCl | NaOCl | | |
| Aqueous | — | — | — | 3.3% by | 16.7% by |
| hydrogen | | | | mass of | mass of |
| peroxide | | | | H₂O₂ | H₂O₂ |
| solution at | | | | | |
| 35% by | | | | | |
| mass | | | | | |
| pH product | 6.8 | 6.4 | 6.0 | 6.2 | 6.0 |
| Visual | Homogeneous | Homogeneous | Homogeneous | Homogeneous | Homogeneous |
| appearance | liquid | liquid | liquid | liquid | liquid |
| product | | | | | |

### 4) Evaluation of the Foaming Properties

### 4.1) Principle of the Evaluation Method

The evaluation of the foaming properties of the test compositions is performed by forming a foam, from a solution of WHO hard water comprising a predetermined mass content of the test compositions, by mechanical stirring at a temperature of 20° C.

4.2) Experimental Protocol

250 cm³ aqueous solutions are prepared so as to obtain solutions at 0.5% by mass of surfactant active material in WHO hard water, from the compositions E₀ to E₄, and F₀ to F₂₄, as described above.

The WHO hard water contains, per liter of deionized water, 0.403 g of anhydrous calcium chloride and 0.139 g of magnesium chloride hexahydrate; which gives it a hardness titer equal to 34°Th.

These solutions are poured into a 500 cm³ beaker and are then stirred using a Rayneri™ laboratory blender (model 33/300) equipped with a butterfly paddle with three hollow arms, at a constant speed of 3000 rpm for 2 minutes.

### 4.3) Expression of the Results

The following parameters are measured for each test:
The expansion time (T_{exp.}): this is the stirring time after which suppression of the vortex in the beaker is observed. Beyond this time, the foam totally surrounds the shaft of the paddle and its level is horizontal;
The half-life time (T_{1/2}): this is the time after which the foam obtained from a certain volume of foaming solution became drained of an amount of solution corresponding to half of the initial volume. For this test, the half-life time is reached when the upper level of the draining water reaches the 125 cm³ mark on the beaker;
The height of foam generated by stirring (Hₜ₀): this is the height of foam generated at the end of the 2 minutes of stirring;
The residual foam height after 30 minutes (Hₜ₃₀): this is the foam height observed 30 minutes after the end of the 2 minutes of stirring.
The difference Δ_{H}=(Hₜ₀−Hₜ₃₀), makes it possible to evaluate comparatively the quality of the foams generated by the various surfactants.

### 4.4) Results Obtained

The results obtained for the aqueous solutions of active material in WHO hard water for the compositions of the compositions E₀ to E₆, and F₀ to F₂₄ are shown in tables 10 to 15 below.

TABLE 10

| | | | | |
|---|---|---|---|---|
| (Tₑₓₚ) | (T_{1/2}) | (Hₜ₀) | (Hₜ₃₀) | (ΔH) |

| | | | | | | | | |
|---|---|---|---|---|---|---|---|---|
| E₀ | 18 | s | >240 | min | 125 mm | 120 mm | 5 | mm |
| E₁ | 7 | s | >240 | min | 140 mm | 130 mm | 10 | mm |
| E₂ | 6 | s | >240 | min | 140 mm | 130 mm | 10 | mm |
| E₃ | 17 | s | >240 | min | 140 mm | 130 mm | 10 | mm |
| E₄ | 22 | s | >240 | min | 140 mm | 130 mm | 10 | mm |
| E₅ | 6 | s | >240 | min | 132 mm | 130 mm | 2 | mm |
| E₆ | 8 | s | >240 | min | 134 mm | 134 mm | 0 | mm |
| E₇ | 10 | s | 155 | min | 140 mm | 125 mm | 15 | mm |
| E₈ | 16 | s | 120 | min | 152 mm | 130 mm | 22 | mm |

TABLE 11

| | | | | |
|---|---|---|---|---|
| (Tₑₓₚ) | (T_{1/2}) | (Hₜ₀) | (Hₜ₃₀) | (ΔH) |

| | | | | | | | |
|---|---|---|---|---|---|---|---|
| F₀ | 12 | s | >240 | min | <100 mm | <100 mm | — |
| F₁ | 8 | s | >240 | min | <100 mm | <100 mm | — |
| F₂ | 9 | s | 150 | min | <100 mm | <100 mm | — |
| F₃ | 9 | s | >240 | min | <100 mm | <100 mm | — |
| F₄ | 8 | s | >240 | min | <100 mm | <100 mm | — |

TABLE 12

| | (Tₑₓₚ) | | (T_{1/2}) | (Hₜ₀) | | (Hₜ₃₀) | | (ΔH) |
|---|---|---|---|---|---|---|---|---|
| F₅ | >120 | s | >240 min | 80 | mm | <80 | mm | — |
| F₆ | 35 | s | >240 min | 110 | mm | <100 | mm | — |
| F₇ | >120 | s | >240 min | <100 | mm | <100 | mm | — |
| F₈ | >120 | s | >240 min | <100 | mm | <100 | mm | — |
| F₉ | >120 | s | >240 min | <100 | mm | <100 | mm | — |

TABLE 13

| | (Tₑₓₚ) | | (T_{1/2}) | (Hₜ₀) | (Hₜ₃₀) | (ΔH) |
|---|---|---|---|---|---|---|
| F₁₀ | >120 | s | >240 min | <100 mm | <100 mm | — |
| F₁₁ | 35 | s | >240 min | <100 mm | <100 mm | — |
| F₁₂ | >120 | s | >240 min | <100 mm | <100 mm | — |
| F₁₃ | >120 | s | >240 min | <100 mm | <100 mm | — |
| F₁₄ | >120 | s | >240 min | <100 mm | <100 mm | — |

TABLE 14

| | (Tₑₓₚ) | | (T_{1/2}) | | (Hₜ₀) | | (Hₜ₃₀) | | (ΔH) |
|---|---|---|---|---|---|---|---|---|---|
| F₁₅ | 50 | s | >240 | min | 90 | mm | 65 | mm | 25 mm |
| F₁₆ | >120 | s | >240 | min | <100 | mm | <100 | mm | — |
| F₁₇ | >120 | s | >240 | min | <100 | mm | <100 | mm | — |
| F₁₈ | 80 | s | 180 | min | 80 | mm | 55 | mm | 25 mm |
| F₁₉ | 60 | s | 180 | min | 75 | mm | 50 | mm | 25 mm |

TABLE 15

| | (Tₑₓₚ) | (T_{1/2}) | | (Hₜ₀) | (Hₜ₃₀) | (ΔH) |
|---|---|---|---|---|---|---|
| F₂₀ | 7 s | 180 | min | 138 mm | 126 mm | 12 mm |
| F₂₁ | 4 s | 120 | min | 144 mm | 129 mm | 15 mm |
| F₂₂ | 5 s | 105 | min | 144 mm | 128 mm | 16 mm |
| F₂₃ | 6 s | 140 | min | 135 mm | 125 mm | 10 mm |
| F₂₄ | 4 s | 20 | min | 168 mm | 160 mm | 25 mm |

### 4.5) Analyses of the Results

These results show that the compositions E₁ to E₆ according to the invention make it possible to prepare foams having all the qualities required to be used in a process for decontaminating hard surfaces, because they are generated rapidly, in a sufficient volume (>100 mm), and stable (with a half-life of over four hours).

1. A disinfecting composition (C_{D}) suitable for topical use comprising, per 100% of mass
a)—from 35% to 99.3% by mass of water;
b)—from 0.5% to 40% by mass of at least one disinfectant;
c)—from 0.1% to 10% by mass of at least one gelling agent and/or thickener (AG);
d)—from 0.1% to 15% by mass of a mixture (M₁) comprising, per 100% of the mixture's own mass:
(i)—from 50% to 99% by mass of a composition (C₁) comprising, per 100% of mass of the composition (C₁):
(α)—from 65% to 90% by mass of at least one compound of formula (I):
R₁—C(═O)—NH—CH(COOH)—(CH₂)₂—COOH (I)
in acid, partially salified or totally salified form, wherein the group R₁—C(═O)— represents a linear or branched, saturated or unsaturated acyl radical including from 8 to 18 carbon atoms, and
(β)—from 10% to 35% by mass of at least one compound of formula (II):
R₁—C(═O)—OH (II)
in acid, partially salified or totally salified form, wherein the group R₁ is as defined for formula (I),
(ii)—from 1% to 50% by mass of a composition (C₂) comprising, per 100% of its mass:
(γ)—from 14% to 80% by mass of a composition (C₃) or of a mixture of compositions (C₃), said composition (C₃) being represented by formula (III):
R₃—O-(G₃)ₚ-H (III)
wherein R₃ represents a linear or branched, saturated or unsaturated aliphatic radical including from 12 to 16 carbon atoms, G₃ represents the residue of a reducing sugar and p represents a decimal number greater than or equal to 1.05 and less than or equal to 5;
(δ)—from 20% to 80% of a composition (C₄) or of a mixture of compositions (C₄), said composition (C₄) being represented by formula (V):
R₄—O-(G₄)_{q}-H (V)
wherein R₄ represents a linear aliphatic radical, chosen from n-butyl (n-C₄H₉—), n-pentyl (n-C₅H₁₁—), n-hexyl (n-C₆H₁₃—) and n-heptyl (n-C₇H₁₅—) radicals, G₄ represents a reducing sugar residue and q represents a decimal number greater than or equal to 1.05 and less than or equal to 5.

2. The disinfecting composition (C_{D}) as claimed in claim 1, wherein the gelling agent and/or thickener (AG) is chosen from polysaccharides consisting of monosaccharide derivatives, polysaccharides consisting solely of monosaccharides, cellulose and cellulose derivatives, starches and linear or branched or crosslinked polyelectrolytes.

3. The disinfecting composition (C_{D}) as claimed in claim 1, wherein the composition (C₃) consists of a mixture of compounds represented by formulae (III₁), (III₂), (III₃), (III₄) and (III₅):
R₃—O-(G₃)₁-H (III₁),
R₃—O-(G₃)₂-H (III₂),
R₃—O-(G₃)₃-H (III₃),
R₃—O-(G₃)₄-H (III₄),
R₃—O-(G₃)₅-H (III₅),
in the respective molar proportions a₁, a₂, a₃, a₄ and a₅ such that:
the sum a₁+a₂+a₃+a₄+a₅ is equal to 1, and
the sum a₁+2a₂+3a₃+4a₄+5a₅ is equal to p.

4. The disinfecting composition (C_{D}) as claimed in claim 1, wherein the composition (C₄) consists of a mixture of compounds represented by formulae (V₁), (V₂), (V₃), (V₄) and (V₅):
R₄—O-(G₄)₁-H (V₁),
R₄—O-(G₄)₂-H (V₂),
R₄—O-(G₄)₃-H (V₃),
R₄—O-(G₄)₄-H (V₄),
R₄—O-(G₄)ₛ-H (V₅),
in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q.

5. The disinfecting composition (C_{D}) as claimed in claim 1, wherein the composition (C₂) comprises:
from 0% to 3% by mass of at least one alcohol of formula (IV):
R₃—OH (IV)
wherein R₃ is as defined in formula (III), and/or
from 0% to 3% by mass of at least one alcohol of formula (VI):
R₄—OH (VI)
wherein R₄ is as defined for formula (V).

6. The disinfecting composition (C_{D}) as claimed in claim 1, wherein, in formulae (I) and (II), the group R₁—C(═O)— represents an acyl radical chosen from octanoyl, decanoyl, dodecanoyl, tetradecanoyl, hexadecanoyl, octadecanoyl, 9-octadecenoyl, 9,12-octadecadienoyl and 9,12,15-octadecatrienoyl radicals.

7. The disinfecting composition (C_{D}) as claimed in claim 1, wherein the composition (C₂) comprises, per 100% of mass:
(γ)—a mass proportion of said composition (C₃) of greater than or equal to 14% and less than 70%, and
(δ)—a mass proportion of said alcohol of formula (IV) of greater than or equal to 0% and less than or equal to 3%,
(ε)—a mass proportion of said composition (C₄) of greater than or equal to 30% and less than or equal to 80%, and
(η)—a mass proportion of said alcohol of formula (VI) of greater than or equal to 0% and less than or equal to 3%.

8. The disinfecting composition (C_{D}) as claimed in claim 1, wherein in formulae (III) and (IV), R₃ represents a linear alkyl radical chosen from n-dodecyl (n-C₁₂H₂₅—), n-tetradecyl (n-C₁₄H₂₉—) and n-hexadecyl (n-C₁₆H₃₂—) radicals.

9. The disinfecting composition (C_{D}) as claimed in claim 1, wherein, in formulae (V) and (VI), R₄ represents a linear alkyl radical chosen from n-hexyl (n-C₆H₁₃) and n-heptyl (n-C₇H₁₅) radicals.

10. The disinfecting composition (C_{D}) as claimed in claim 1, wherein the composition (C₂) comprises a mixture of compositions (C₃) and compositions (C₄), said mixture comprising, per 100% of mass:
(γ₁)—from 13.6% to 44.4% by mass of a composition (C₃) represented by formula (III) wherein R₃ represents the (n-C₁₂H₂₅) radical,
(γ₂)—from 5% to 16.25% by mass of a composition (C₃) represented by formula (III) wherein R₃ represents the n-tetradecyl (n-C₁₄H₂₉) radical, and
(γ₃)—from 1.4% to 4.55% by mass of a composition (C₃) represented by formula (III) wherein R₃ represents the n-hexadecyl (n-C₁₆H₃₂) radical,
(ε₁)—from 35% to 80% by mass of a composition (C₄) represented by formula (V) wherein R₄ represents the n-heptyl radical (n-C₇H₁₅) radical.

11. The disinfecting composition (C_{D}) as claimed in claim 1, wherein the mass ratio:
Δ=Mass of compound(s) of formula (I)/[Mass of composition (C₃)+Mass of composition (C₄)], is greater than or equal to 20/80 and less than or equal to 65/35.

12. The disinfecting composition (C_{D}) as claimed in claim 1, wherein that the mass ratio:
Δ₁=Mass of composition (C₃)/Mass of composition (C₄) is greater than or equal to 20/80 and less than or equal to 70/30.

13. The disinfecting composition (C_{D}) as claimed in claim 1, wherein at least one gelling agent and/or thickener (AG) is chosen from xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to ⅓ and less than or equal to 3/1.

14. The disinfecting composition (C_{D}) as claimed in claim 1, wherein at least disinfectant is chosen from the elements of the group consisting of sorbic acid, sodium sorbate, potassium sorbate, dehydroacetic acid, sodium dehydroacetate, benzoic acid, sodium benzoate, potassium benzoate; 1-(2-ethylhexyl) glycerol; hydrogen peroxide; activated peroxides, mixtures of hydrogen peroxide and urea, mixtures of hydrogen peroxide and peracetic acid and mixtures of hydrogen peroxide and iron (Fenton's reagent); hydroperoxycarbonates; peracetic acid; sodium hypochlorite.

15. The disinfecting composition (C_{D}) as claimed in claim 1, further comprising, per 100% of mass:
a)—from 35% to 99.3% water;
b)—from 0.5% to 40% by mass of at least one disinfectant chosen from the group consisting of hydrogen peroxide, sodium hypochlorite, potassium sorbate, sodium benzoate, 1-(2-ethylhexyl) glycerol,
c)—from 0.1% to 10% by mass of at least one gelling agent and/or thickener (AG) chosen from the elements of the group consisting of xanthan gum (G_{X}), acacia gum exudate (G_{A}), the mixture of xanthan gum (G_{X}) and of acacia gum exudate (G_{A}) in a mass ratio between the xanthan gum (G_{X}) and the acacia gum exudate (G_{A}) which is greater than or equal to ⅓ and less than or equal to 3/1;
d)—from 0.1% to 15% by mass of said mixture (M₁) wherein:
the compound of formula (I) is chosen from monosodium N-cocoyl glutamate, monopotassium N-cocoyl glutamate, disodium N-cocoyl glutamate and dipotassium N-cocoyl glutamate,
the compound of formula (II) is chosen from sodium cocoate and potassium cocoate,
in formula (III), R₃ represents a linear or branched, saturated or unsaturated aliphatic radical including from 12 to 16 carbon atoms, G₃ represents a glucose or xylose residue and p represents a decimal number greater than or equal to 1.05 and less than or equal to 2.5;
in formula (IV), R₃ represents a linear or branched, saturated or unsaturated aliphatic radical including from 12 to 16 carbon atoms,
in formula (V), R₄ represents the n-heptyl (n-C₇H₁₅) radical, G₄ represents a glucose or xylose residue and q represents a decimal number greater than or equal to 1.05 and less than or equal to 2,
in formula (VI), R₄ represents the n-heptyl (n-C₇H₁₅) radical.

16. A method for disinfectant washing of the hands, comprising providing the disinfecting composition of claim 1, and applying an effective amount of the composition to the hands and washing.

17. A method for treating a skin infection, a skin burn, a scar, a wound, in human beings or animals, comprising applying an effective amount of the composition of claim 1 to skin of the human being or animal in need thereof.

18. The disinfecting composition (C_{D}) as claimed in claim 2, wherein the composition (C₃) consists of a mixture of compounds represented by formulae (III₁), (III₂), (III₃), (III₄) and (III₅):
R₃—O-(G₃)₁-H (III₁),
R₃—O-(G₃)₂-H (III₂),
R₃—O-(G₃)₃-H (III₃),
R₃—O-(G₃)₄-H (III₄),
R₃—O-(G₃)₅-H (III₅),
in the respective molar proportions a₁, a₂, a₃, a₄ and a₅ such that:
the sum a₁+a₂+a₃+a₄+a₅ is equal to 1, and
the sum a₁+2a₂+3a₃+4a₄+5a₅ is equal to p.

19. The disinfecting composition (C_{D}) as claimed in claim 2, wherein the composition (C₄) consists of a mixture of compounds represented by formulae (V₁), (V₂), (V₃), (V₄) and (V₅):
R₄—O-(G₄)₁-H (V₁),
R₄—O-(G₄)₂-H (V₂),
R₄—O-(G₄)₃-H (V₃),
R₄—O-(G₄)₄-H (V₄),
R₄—O-(G₄)ₛ-H (V₅),
in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q.

20. The disinfecting composition (C_{D}) as claimed in claim 3, wherein the composition (C₄) consists of a mixture of compounds represented by formulae (V₁), (V₂), (V₃), (V₄) and (V₅):
R₄—O-(G₄)₁-H (V₁),
R₄—O-(G₄)₂-H (V₂),
R₄—O-(G₄)₃-H (V₃),
R₄—O-(G₄)₄-H (V₄),
R₄—O-(G₄)ₛ-H (V₅),
in the respective molar proportions a′₁, a′₂, a′₃, a′₄ and a′₅, such that:
the sum a′₁+a′₂+a′₃+a′₄+a′₅ is equal to 1, and
the sum a′₁+₂a′₂+3a′₃+4a′₄+5a′₅ is equal to q.
